(19) United States
(12) Patent Application Publication
(10) Pub. No.: US 2023/0137861 A1
(43) Pub. Date: May 4, 2023

(54) BIOMARKER COMPOSITION FOR DIAGNOSING DIFFUSE TYPE GASTRIC CANCER, AND MEDICAL USE THEREOF

(71) Applicant: AJOU UNIVERSITY INDUSTRY-ACADEMIC COOPERATION FOUNDATION, Suwon-si (KR)

(72) Inventors: Hoon HUR, Seoul (KR); In-hye HAM, Suwon-si (KR); Dagyeong LEE, Suwon-si (KR)

(73) Assignee: AJOU UNIVERSITY INDUSTRY-ACADEMIC COOPERATION FOUNDATION, Suwon-si (KR)

(21) Appl. No.: 17/907,791

(22) PCT Filed: Feb. 24, 2021
(86) PCT No.: PCT/KR2021/002276

§ 371 Date: Aug. 29, 2022

(30) Foreign Application Priority Data
Feb. 28, 2020 (KR) 10-2020-0024778
Feb. 3, 2021 (KR) 10-2021-0015216

(51) Int. Cl. C12Q 1/6886; G01N 33/574

(52) U.S. Cl. CPC C12Q 1/6886; G01N 33/57446; C12Q 1/6851

(57) ABSTRACT

The present invention relates to: a biomarker composition for diagnosing diffuse type gastric cancer, containing, as an active ingredient, a TINAGL1 protein or a gene encoding the protein; and a medical use thereof. More specifically, it is ascertained that: the secretion of TINAGL1 increases in cancer-associated fibroblasts separated from a diffuse type gastric cancer tissue; TINAGL1 improves metastasis and tumor formation of diffuse type gastric cancer by activating FAK signaling of cancer cells; the increase in TINAGL1 expression is related to the prognosis of a diffuse type gastric cancer patient. Therefore, TINAGL1 can be provided as a biomarker for the diagnosis and prognosis prediction of diffuse type gastric cancer, and a TINAGL1 expression or activity inhibitor can be provided as an effective agent for treating diffuse type gastric cancer.

### TECHNICAL FIELD

The present disclosure relates to a biomarker composition for diagnosing diffuse type gastric cancer including TINAGL1 protein or a gene encoding the protein as an active ingredient, and to a medical use thereof.

### BACKGROUND ART

Gastric cancer is the most common cancer in Korea, but as gastroscopy became more common and gastric cancer was detected at an early stage, the 5-year survival rate of gastric cancer patients (the proportion of patients who were diagnosed with cancer and survived more than 5 years) improved to 75.4%. As such, although the early detection rate of gastric cancer is increasing, diffuse type gastric cancer with a poor prognosis accounts for 40% of all gastric cancers.

Gastric cancer is divided into intestinal type gastric cancer and diffuse type gastric cancer, wherein in intestinal type gastric cancer, the cancer grows in the form of a lump on the mucosal surface, and the growth rate is slower than that of the diffuse type. On the other hand, in diffuse type gastric cancer, changes in the surface of the mucosa are not clearly seen, and precancerous lesions are not easily identified like adenomas because cancer cells that are small enough to be invisible penetrate the stomach wall and grow under the mucous membrane. However, the proliferation rate is fast, and when cancer is discovered, it is often already advanced and most are found at stage 3˜4, so the prognosis is poor. In addition, the possibility of distant metastasis is high, and it easily metastasizes to adjacent sites such as the esophagus or duodenum.

In particular, as for gastric cancer occurring at a young age, diffuse type gastric cancer which progresses and metastasizes quickly, accounts for 60˜70% of gastric cancer, requiring special attention from young people to diffuse type gastric cancer.

The treatment of diffuse type gastric cancer is not significantly different from intestinal type gastric cancer, and is determined in consideration of the patient's condition and disease progression. For diffuse type gastric cancers detected at an early stage, in the case of lesions that do not have lymph node metastasis and are treatable locally, endoscopic submucosal dissection can be performed to remove only the cancer cells without resection of the stomach to minimize complications and sequelae, and a cure rate similar to that of surgery can be expected. Accordingly, early detection of diffuse gastric cancer is of utmost importance.

According to the current gastric cancer screening recommendations in Korea, while gastroscopy is recommended every two years for those over 40 years of age, diffuse type gastric cancer is not easy to diagnose with endoscopy alone and thus additional examinations such as abdominal CT scan and endoscopic ultrasound are required.

### DISCLOSURE

### Technical Goals

As described above, diffuse type gastric cancer can be expected to have a high cure rate through early detection, but it is difficult to diagnose with general endoscopic ultrasound. To solve this issue, the present disclosure provides TINAGL1 which is secreted and expressed in cancer-associated fibroblast (CAF) in gastric cancer tissue as a biomarker composition for diagnosing diffuse type gastric cancer.

### Technical Solutions

Example embodiments of the present disclosure provide a biomarker composition for diagnosing diffuse type gastric cancer including TINAGL1 protein or a gene encoding the protein as an active ingredient.

Example embodiments of the present disclosure provide a kit for diagnosing diffuse type gastric cancer including, as an active ingredient, an agent capable of detecting an expression or activity level of TINAGL1 protein, or an expression level of a gene encoding the protein.

Example embodiments of the present disclosure provide a method of providing information for diagnosis of diffuse type gastric cancer, including comparing an expression or activity level of TINAGL1 protein, or an expression level of a gene encoding the protein of a sample isolated from a specimen and that of a sample isolated from a normal control.

Example embodiments of the present disclosure provide a biomarker composition for predicting prognosis of diffuse type gastric cancer including TINAGL1 protein or a gene encoding the protein as an active ingredient.

Example embodiments of the present disclosure provide a pharmaceutical composition for preventing or treating diffuse type gastric cancer including a TINAGL1 inhibitor as an active ingredient.

Example embodiments of the present disclosure provide a pharmaceutical composition for preventing or treating diffuse type gastric cancer including a TINAGL1 inhibitor and an anticancer agent as active ingredients.

In addition, example embodiments of the present disclosure provide a drug screening method for treatment of diffuse type gastric cancer including determining a TINAGL1 expression level by culturing cancer-associated fibroblasts (CAF) isolated from diffuse gastric cancer tissue (step 1), determining a TINAGL1 expression level by treating the TINAGL1-expressed CAF with a candidate material (step 2), and comparing the TINAGL1 expression level of step 1 with the TINAGL1 expression level of step 2 (step 3).

### Advantageous Effects

According to the present disclosure, it was found that TINAGL1 secretion was increased in CAF isolated from diffuse type gastric cancer tissue and the TINAGL1 activated an FAK signal of cancer cells to improve metastasis and tumor formation of diffuse type gastric cancer, and that the increase in TINAGL1 expression is related to the prognosis of patients with diffuse type gastric cancer. Accordingly, it is possible to provide the TINAGL1 as a biomarker for diagnosis of diffuse type gastric cancer and predicting its prognosis, and to provide an inhibitor of TINAGL1 expression or activity as an effective therapeutic agent for diffuse type gastric cancer.

### BRIEF DESCRIPTIONS OF THE DRAWINGS

FIG. 1 shows results confirming effects of cancer-associated fibroblasts (CAF) and normal fibroblasts (NAF) on cancer cells, from which migration and metastatic abilities of cancer cells in SNU668 cells treated with CAF or NAF are confirmed.

FIG. 2 shows results confirming effects of cancer-associated fibroblasts (CAF) and normal fibroblasts (NAF) on cancer cells, from which tumorigenicity and migration abilities of cancer cells in SNU601 cells treated with CAF or NAF are confirmed.

FIG. 3 shows results of transcriptome analysis and proteomics analysis between cancer-associated fibroblasts (CAF) and normal fibroblasts (NAF).

FIG. 4 shows results of quantitative RT-PCR, Western blot, real-time PCR and ELISA assay confirming differences in TINAGL1 expression levels in CAF and NAF isolated from patients with diffuse type gastric cancer.

FIG. 5 shows results of Western blot analysis confirming effects of cancer-associated fibroblasts (CAF) and normal fibroblasts (NAF) on FAK signal activation in diffuse type gastric cancer cells.

FIG. 6 shows results of Western blot analysis confirming FAK signaling activity of a diffuse type gastric cancer cell line treated with recombinant TINAGL1.

FIG. 7 shows results confirming tumorigenicity of a gastric cancer cell line SNU601 treated with recombinant TINAGL1.

FIG. 8 shows results of Western blot and RT-PCR analysis confirming cancer-associated fibroblasts (CAF) in which TINAGL1 expression is inhibited by transfection with TINAGL1 siRNA.

FIG. 9 shows results of immunocytochemical staining confirming cancer-associated fibroblasts (CAF) in which TINAGL1 expression is inhibited by transfection with TINAGL1 siRNA.

FIG. 10 shows a result confirming effects of TINAGL1 expression inhibition on a migratory ability of gastric cancer cells after co-culturing cancer-associated fibroblasts (CAF) in which TINAGL1 expression is inhibited with gastric cancer cells.

FIG. 11 shows results of Western blot analysis confirming an effect of TINAGL1 expression inhibition on gastric cancer cell signaling after co-culturing cancer-associated fibroblasts (CAF) in which TINAGL1 expression is inhibited with gastric cancer cells.

FIG. 12 shows results of Western blot analysis confirming an expression level of TINAGL1 in cancer tissues and normal gastric tissues isolated from patients with diffuse type gastric cancer.

FIG. 13 shows results confirming the expression levels of TINAGL1 and COL1A1 in cancer tissues and normal gastric tissues isolated from patients with diffuse type gastric cancer, from which the mRNA expression levels of COL1A1 (red, fibroblast marker) and TINAGL1 (green) in formalin-fixed paraffin-embedded (FFPE) tissues of patients with diffuse type gastric cancer are confirmed.

FIG. 14 shows results confirming the prognosis of patients with diffuse type gastric cancer according to the TINAGL1 expression levels.

### BEST MODE

Hereinafter, the present invention will be described in more detail.

Among various histological subtypes of gastric cancer, diffuse type gastric cancer is known to have a poor prognosis. As cancer-associated fibroblasts accumulated in diffuse type gastric cancer are reported to be related to the prognosis of patients, the inventors completed the present invention by confirming that TINAGL1 secretion was increased in cancer-associated fibroblasts isolated from diffuse type gastric cancer tissue, and that TINAGL1 activated the FAK signal of cancer cells to improve metastasis and tumor formation of diffuse typegastric cancer.

Example embodiments of the present disclosure provide a biomarker composition for diagnosing diffuse type gastric cancer including TINAGL1 protein or a gene encoding the protein as an active ingredient.

In detail, the “TINAGL1” is “Tubulointerstitial nephritis antigen-like 1” (Gene ID: 64129; GenBank: AAH09048.1).

The secretion and expression of the TINAGL1 may increase in cancer-associated fibroblasts (CAF) in gastric cancer tissue.

Example embodiments of the present disclosure provide a kit for diagnosing diffuse type gastric cancer including, as an active ingredient, an agent capable of detecting an expression or activity level of TINAGL1 protein, or an expression level of a gene encoding the protein.

The agent may be selected from the group consisting of a primer, a probe, an antibody, a peptide, and an aptamer.

In addition, example embodiments of the present disclosure provide a method of providing information for diagnosis of diffuse type gastric cancer, including comparing an expression or activity level of TINAGL1 protein, or an expression level of a gene encoding the protein of a sample isolated from a specimen and that of a sample isolated from a normal control.

The term “biomarker” as used herein refers to a substance capable of diagnosing by distinguishing a tissue or cell of an individual with gastric cancer from a tissue or cell of a normal control, and includes organic biomolecules such as proteins or nucleic acids, lipids, glycolipids, glycoproteins, and the like, showing an increase or decrease in tissue or cells of the individual having the disease compared to the normal control.

In addition, example embodiments of the present disclosure provide a biomarker composition for predicting prognosis of diffuse type gastric cancer including TINAGL1 protein or a gene encoding the protein as an active ingredient.

The term “diagnosis” as used herein broadly refers to determining the actual condition of a patient's disease in all aspects. The determination includes a disease name, an etiology, a disease type, severity, a detailed mode of the disease, and the presence or absence of complications.

The term “prognosis” as used herein includes determining whether an individual, i.e., a test subject, for a specific disease or condition will develop the disease in the future, or determining responsiveness of the test subject to treatment.

In addition, example embodiments of the present disclosure provide a pharmaceutical composition for preventing or treating diffuse type gastric cancer including a TINAGL1 inhibitor as an active ingredient.

The TINAGL1 inhibitor may inhibit secretion and expression of TINAGL1 protein or a gene encoding the protein in CAF in diffuse type gastric cancer tissue.

The TINAGL1 inhibitor may be any one selected from the group consisting of a peptide, an aptamer, an antibody, a antisense nucleotide, a siRNA, a shRNA, and a miRNA that specifically bind to the TINAGL1 protein or a gene encoding the protein.

In addition, example embodiments of the present disclosure provide a pharmaceutical composition for preventing or treating diffuse type gastric cancer including a TINAGL1 inhibitor and an anticancer agent as active ingredients.

The anticancer agent may be selected from the group consisting of doxorubicin, paclitaxel, vincristine, daunorubicin, vinblastine, actinomycin-D, docetaxel, etoposide, teniposide, bisantrene, imatinib, cisplatin, and 5-fluorouracil.

In an example embodiment of the present disclosure, the pharmaceutical composition including the TINAGL1 inhibitor as an active ingredient may use any one formulation selected from the group consisting of injections, granules, powders, tablets, pills, capsules, suppositories, gels, suspensions, emulsions, drops, or liquids according to a conventional method.

In another example embodiment of the present disclosure, the pharmaceutical composition including the TINAGL1 inhibitor as an active ingredient may further include one or more additives selected from the group consisting of suitable carriers, excipients, disintegrants, sweeteners, coating agents, swelling agents, lubricants, flavoring agents, antioxidants, buffers, bacteriostats, diluents, dispersants, surfactants, binders and lubricants commonly used in preparation of pharmaceutical compositions.

Specifically, as the carriers, excipients, and diluents, lactose, dextrose, sucrose, sorbitol, mannitol, xylitol, erythritol, maltitol, starch, gum acacia, alginate, gelatin, calcium phosphate, calcium silicate, cellulose, methyl cellulose, microcrystalline cellulose, polyvinyl pyrrolidone, water, methylhydroxybenzoate, propylhydroxybenzoate, talc, magnesium stearate, and mineral oil may be used. The solid preparations for oral administration include tablets, pills, powders, granules, capsules, etc., and such solid preparations may be prepared by mixing at least one excipient, for example, starch, calcium carbonate, sucrose or lactose, gelatin, and the like, with the composition. In addition to simple excipients, lubricants such as magnesium stearate and talc may also be used. Liquid formulations for oral use may include suspensions, solutions, emulsions, syrups, and the like, and in addition to water and liquid paraffin, which are commonly used simple diluents, various excipients, for example, wetting agents, sweetening agents, fragrances, preservatives, and the like may be included. Formulations for parenteral administration include sterile aqueous solutions, non-aqueous solvents, suspensions, emulsions, freeze-dried preparations, suppositories, and the like. As non-aqueous solvents and suspensions, propylene glycol, polyethylene glycol, vegetable oils such as olive oil, and injectable esters such as ethyl oleate may be used. As a base material for the suppository, witepsol, macrogol, tween 61, cacao butter, laurin, glycerogelatin, and the like may be used.

According to an example embodiment of the present disclosure, the pharmaceutical composition may be administered to a subject in a conventional manner via intravenous, intraarterial, intraperitoneal, intramuscular, intrasternal, transdermal, intranasal, inhalation, topical, rectal, oral, intraocular or intradermal routes.

The preferred dosage of the TINAGL1 inhibitor may vary depending on the condition and weight of the subject, the type and extent of the disease, the drug form, the administration route and duration, and may be appropriately selected by those skilled in the art. According to an example embodiment of the present disclosure, the daily dose may be 0.01 to 200 mg/kg, specifically 0.1 to 200 mg/kg, and more specifically 0.1 to 100 mg/kg, but not limited thereto. Administration may be done once a day or may be divided into several doses, but the scope of the present disclosure is not limited thereto.

The term “individual” or “subject” as used herein may refer to a mammal including a human, but not limited thereto.

In addition, an example embodiment of the present disclosure provides a drug screening method for treatment of diffuse type gastric cancer including determining a TINAGL1 expression level by culturing cancer-associated fibroblasts (CAF) isolated from diffuse gastric cancer tissue (step 1), determining a TINAGL1 expression level by treating the TINAGL1-expressed CAF with a candidate material (step 2), and comparing the TINAGL1 expression level of step 1 with the TINAGL1 expression level of step 2 (step 3).

The TINAGL1 expression level may be measured by any one or more selected from the group consisting of reverse transcription-polymerase chain reaction (RT-PCR), enzyme-linked immunosorbent assay (ELISA), radioimmunoassay, immunohistochemistry, microarray, western blotting, and flow cytometry (FACS).

### MODES FOR CARRYING OUT INVENTION

Hereinafter, example embodiments of the present disclosure will be described in detail. Examples described below are merely for illustrating the present disclosure in detail, and the scope of the present disclosure is not limited by the examples. The embodiments of the present disclosure are provided to more completely explain the invention to those skilled in the art.

### <Example 1> Confirmation of Difference in Gene Expression Between Cancer-Associated Fibroblasts (CAF) and Normal Fibroblasts (NAF)

Gastric cancer has severe intertumoral heterogeneity according to histological subtypes.

It has been reported that cancer-associated fibroblasts accumulated in diffuse type gastric cancer are associated with the prognosis of patients. In order to identify medical significance of cancer-associated fibroblast (CAF) accumulation in a specific subtype, signet ring cell carcinoma, from the previous study results, stromal collagen or fibroblast marker staining was performed for signet ring cell carcinoma of 175 patients and they were classified into two groups.

As a result, it was determined that one patient group with a large amount of fibrotic matrix showed a poorer prognosis than the other patient group.

From the above study result, the difference in the effect of cancer-associated fibroblast (CAF) and normal gastric tissue-associated fibroblast (NAF) on cancer cells was identified, and transcriptome and proteomic analysis of CAF and NAF was performed.

NAF or CAF was aliquoted in a 24-well plate, and a diffuse type gastric cancer cell line SNU668 was aliquoted in a transwell chamber with a pore size of 8 μm. Thereafter, serum-free DMEM was added to an upper chamber and DMEM supplemented with 10% FBS was added to a lower chamber, and then co-cultured with NAF or CAF or cancer cells were cultured alone.

After 48 hours, the cells were fixed with methanol and H&E staining was performed. After removing the cells present on the top of the membrane in the upper chamber, the membrane was separated from the chamber and mounted with the bottom side up on a slide glass. Cells passing through the membrane were counted by taking three pictures randomly per well at 100 magnification, and then the averages were compared.

A 1.0% agarose gel was made at the bottom of the 24-well plate using agarose (low gelling temperature), and when the gel at the bottom was hardened, SNU668 cancer cells and NAF or CAF were mixed together in 0.5% agarose gel and aliquoted on it to harden.

When the gel at the top was hardened, 300 μl of RPMI with 10% FBS was aliquoted. Thereafter, the medium was replaced with a new medium once every 3 days and observation was made for 20 days.

Four wells were used in each group, and the diameter of colonies was measured in all wells on day 20. The degree of tumorigenesis between the two groups was compared with the average diameter of the top 5 spheroids per well.

In addition, in the same manner as in the previous experiment, SUN601 cells were treated with NAF and CAF, respectively, and cultured together to confirm tumorigenicity and migration ability.

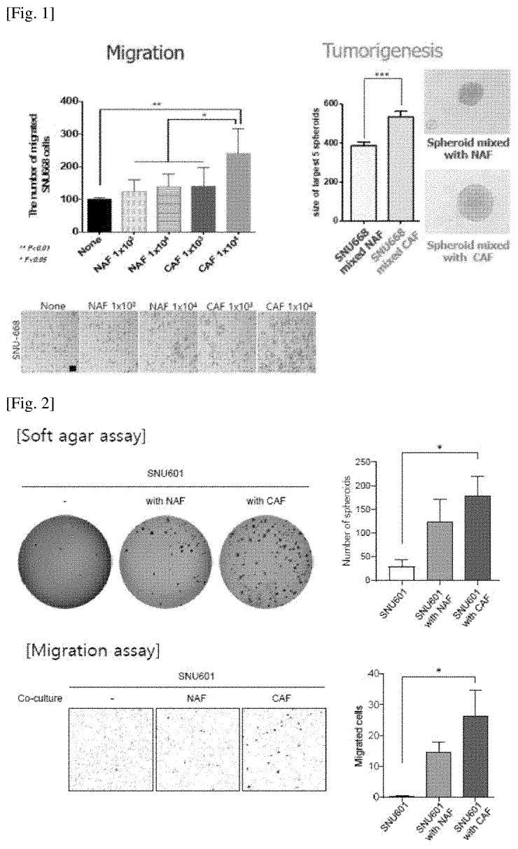

As a result, as shown in FIGS. 1 and 2, it was determined that, in the group co-cultured with CAF, the migrating ability of cancer cells increased more and the average size of the formed tumor spheroids was larger compared to NAF.

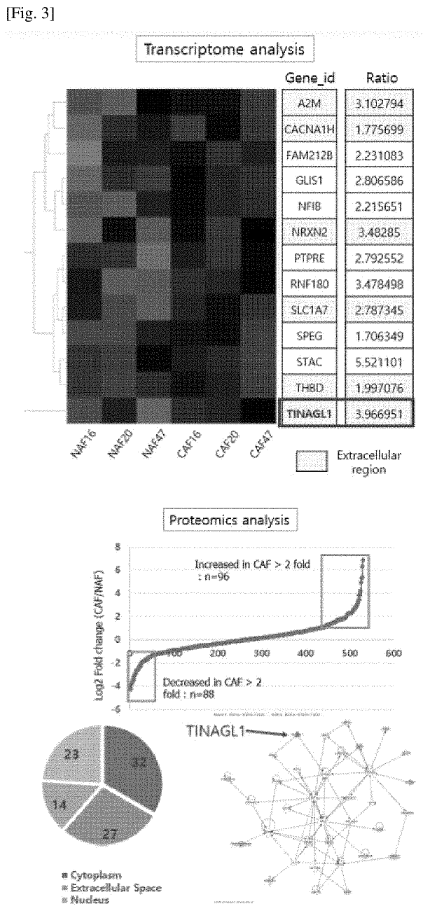

In addition, referring to the transcript analysis result of FIG. 3, 13 genes were generally increased in CAF than in NAF, of which 6 genes were identified as genes related to the extracellular space, and in particular, it was confirmed that TINAGL1 showed the highest ratio.

In addition, referring to the mass spectrometry results, 96 proteins were identified that were significantly increased in CAF-conditioned medium compared to NAF, and as a result of the IPA function analysis of 96 proteins, 27 proteins were classified into a groups related to the extracellular space, of which TINAGL1 was found.

In order to determine whether the TINAGL1, whose expression level was changed, exhibits a difference in expression in CAF and NAF of patients with diffuse type gastric cancer, quantitative RT-PCR, Western blot, real-time PCR, and ELISA were performed.

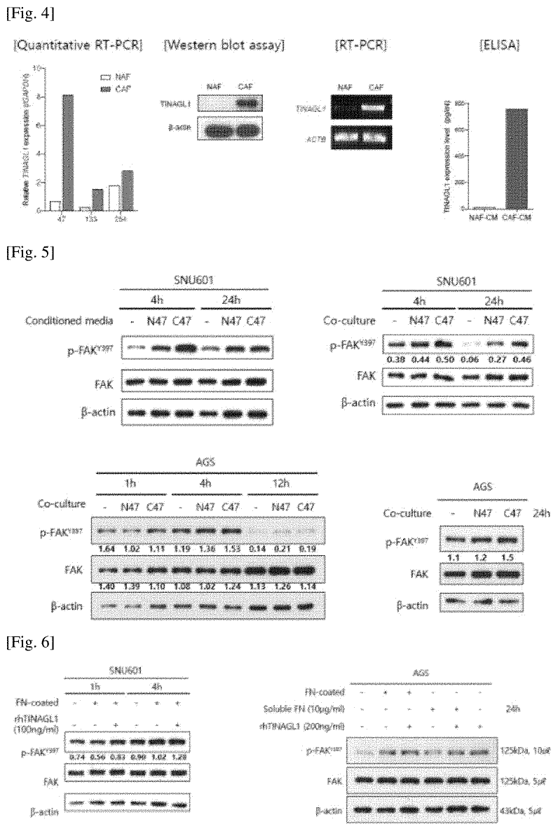

As a result, it was confirmed that TINAGL1 expression was increased in CAF than in NAF of patients with diffuse type gastric cancer as shown in FIG. 4.

An additional experiment was performed to identify relationships between TINAGL1 secreted from CAF confirmed from the above results and gastric cancer progression.

### <Example 2> Confirmation of Effect of Cancer-Associated Fibroblasts (CAF) and Normal Fibroblasts (NAF) on Diffuse Type Gastric Cancer Cell Lines

To determine the effect of cancer-associated fibroblasts (CAF) and normal fibroblasts (NAF) on diffuse type gastric cancer cell lines, Western blot analysis was performed.

First, NAF or CAF was cultured in a 10 cm culture dish with serum-free DMEM for 48 hours. The cultured medium was transferred to a new tube, centrifuged at 4° C. at 2000 rpm for 10 minutes, and the supernatant was collected in a new tube.

Diffuse type gastric cancer cell lines SNU601 (5.5×10⁵) and AGS (3.0×10⁵) were aliquoted in 6-well plates and starvated overnight with serum-free DMEM. After starvation, each cancer cell was cultured in DMEM supplemented with 5% FBS, NAF or CAF conditioned medium.

In addition, cancer cells starvated overnight with serum-free DMEM and NAF or CAF were aliquoted into a 6-well plate and a transwell chamber with a pore size of 0.4 μm, respectively, and co-cultured with DMEM supplemented with 5% FBS.

At each time point, cells were collected, proteins were extracted, electrophoresed by SDS-PAGE, and transferred to PVDF membrane. The PVDF membrane was blocked with 5% skim milk at room temperature for 1 hour.

The primary antibody against the target protein was reacted at 4° C. overnight, and the HRP-conjugated secondary antibody against the primary antibody was reacted for 1 hour at room temperature, visualized using an ECL solution, and the expression of the target protein was normalized with β-actin.

As a result, as shown in FIG. 5, it was confirmed that FAK signaling was activated in cancer cells cultured with NAF or CAF conditioned medium or cultured with NAF or CAF, and that the degree of FAK signaling activation was improved more by CAF than by NAF, and these results were the same in the two cell lines of SNU601 and AGS.

### <Example 3> Confirmation of FAK Signal Activity by TINAGL1

As confirmed in the previous experiment, it was confirmed that CAF activates the FAK signal in diffuse type cancer cells, and accordingly it was determined whether TINAGL1 secreted from CAF could also regulate the FAK signal in diffuse type cancer cells.

The culture dish was coated with fibronectin solution (10 μg/ml in PBS) while incubating at 37° C. for 3 hours.

SNU601 (5.5×10⁵) and AGS (3.0×10⁵) cancer cells were aliquoted in 6-well plates coated with or without fibronectin, and the cancer cells were starvated overnight with serum-free DMEM. After starvation, culture was performed in a medium supplemented with 5% FBS, and fibronectin or recombinant TINAGL1 (100 ng/ml or 200 ng/ml) was directly added to the medium according to each condition.

Thereafter, cells were collected at each time point, proteins were extracted, and Western blot was performed in the same manner as in Example 2 to confirm the degree of FAK signaling activity by fibronectin, one of the ligands of integrin known to be upstream of FAK signaling.

As a result, as shown in FIG. 6, FAK was activated in the fibronectin-treated experimental group, and it was confirmed that the FAK signaling activity was further improved by the addition of TINAGL1. In addition, it was confirmed that FAK signaling was activated even when TINAGL1 was treated alone (last sample on the right).

### <Example 4> Confirmation of Tumorigenicity of TINAGL1

Agarose (low gelling temperature) was used to make a 1.0% agarose gel at the bottom of the 6-well plate, and when the gel at the bottom hardened, SNU601 cancer cells were mixed with 0.5% agarose gel and aliquoted thereon to harden.

When the upper gel was hardened, 500 μl of RPMI (100 ng/ml) supplemented with 10% FBS with or without rhTINAGL1 was aliquoted. Thereafter, it was observed for 20 days by replacing it with a new medium suitable for each once every 3 days.

Three wells were used for each group, and the diameter of colonies was measured in all wells on day 20. The degree of tumorigenesis between the two groups was compared with the average diameter of the top 5 spheroids per well.

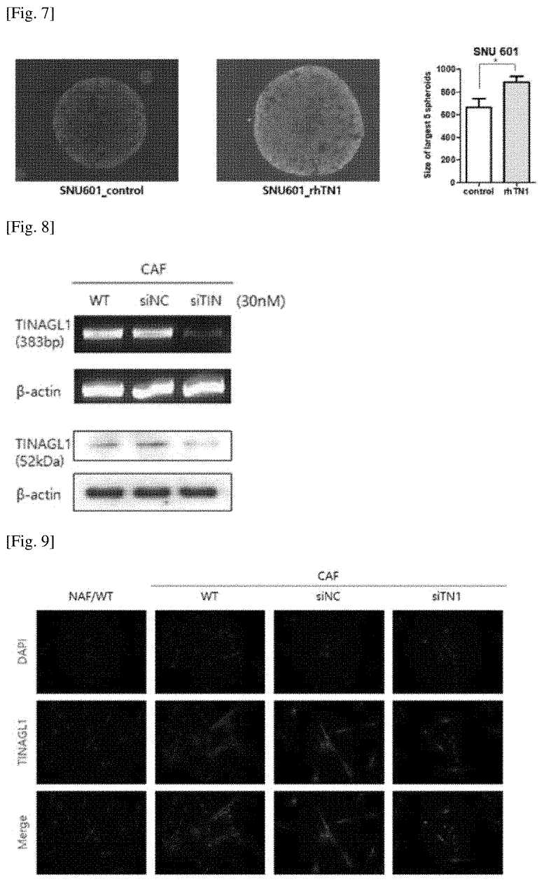

As a result, it was confirmed that the average size of the tumor spheroids formed in the group treated with TINAGL1 was larger than that of the control group as shown in FIG. 7.

### <Example 5> Confirmation of Effect of TINAGL1 Expression-Inhibited CAF on Gastric Cancer Cells

1. Construction of TINAGL1 Expression Inhibited CAFs

Lipofectamine RNAiMAX was used to transfect CAF cells with 30 nM TINAGL1 or NC siRNA for 48 hours. After 48 hours, RNA and protein were prepared, respectively.

After synthesizing cDNA from the prepared RNA, RT-PCR was performed using a primer for the target gene, and the PCR product was confirmed by electrophoresis in 2% agarose gel, 100V for 20 minutes.

In addition, the protein was electrophoresed by SDS-PAGE, and then transferred to a PVDF membrane. Thereafter, Western blot analysis was performed in the same manner as in Example 2.

As a result, as shown in FIG. 8, it was confirmed that the expression of TINAGL1 gene and protein was decreased in CAF transfected with siTINAGL1 (30 nM).

Meanwhile, NAF and CAF were each aliquoted in a 24-well plate at 3×10³ cells/well, and 30 nM siRNA was transfected using Lipofectamine RNAiMAX for 48 hours.

After 48 hours, cells were fixed with 4% paraformaldehyde and blocked with 2% BSA at room temperature for 1 hour. As a primary antibody, TINAGL1 (CUSABIO, 1:100) was bound at 4° C. overnight, and a secondary antibody Cy3-Rabbit (1:500) with fluorescence attached to the primary antibody was bound at room temperature for 2 hours. After processing the mounting solution together with DAPI, it was observed under a microscope.

As a result, more TINAGL1 (red) expression was confirmed in CAF than in NAF, as shown in FIG. 9, and it was confirmed that TINAGL1 expression was decreased in CAF transformed with siTINAGL1.

2. Confirmation of Gastric Cancer Cell Migration Ability by TINAGL1 Expression-Inhibited CAF

CAF was aliquoted in 24-well plates, and 30 nM TINAGL1 or NC siRNA was transformed using Lipofectamine RNAiMAX for 3 days.

SNU601 (2×10⁴) was aliquoted into an 8 μm pored transwell chamber and starvated overnight with serum-free DMEM. Thereafter, serum-free DMEM was added to the upper chamber and DMEM supplemented with 10% FBS was added to the lower chamber, and co-cultured with the transformed NAF or CAF. After 48 hours, cells were fixed with methanol and H&E staining was performed. After removing the cells present on the top of the membrane in the upper chamber, the membrane was separated from the chamber and mounted with the bottom side up on a slide glass. Cells passing through the membrane were counted by taking pictures of three random sites per well at 200 magnification, and then the averages were compared.

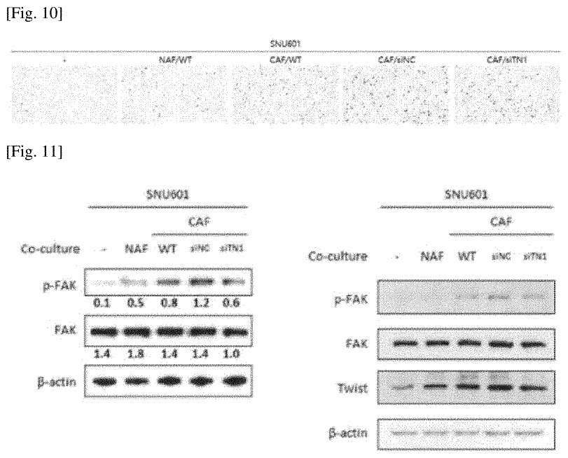

As a result, in the control group culturing SNU601 alone, the migration of cells was hardly observed, as shown in FIG. 10. In the experimental group co-cultured with NAF, cancer cell migration was observed, but it was confirmed that cancer cell migration was significantly increased in the experimental group co-cultured with CAF.

In addition, it was confirmed that cancer cell migration was reduced in the experimental group co-cultured with CAF, in which TINAGL1 expression was suppressed through siRNA transformation, compared to the negative control group.

3. Confirmation of Effect of TINAGL1 Expression-Inhibited CAF on Signaling Mechanism in Gastric Cancer Cells

CAF was aliquoted into a transwell chamber with a pore size of 0.4 μm, and 30 nM TINAGL1 or NC siRNA was transformed using Lipofectamine RNAiMAX for 3 days.

SNU601 was aliquoted into 6-well plates and starvated overnight with serum-free DMEM. Thereafter, the cells were co-cultured with NAF or transformed CAF cells in DMEM supplemented with 5% FBS for 24 hours.

Thereafter, SNU601 cells were collected, proteins were extracted, and Western blot analysis was performed in the same manner as in Example 2.

As a result, FAK signaling increased by CAF co-culture was reduced by TINAGL1 knockdown of CAF as shown in FIG. 11A. In addition, as shown in FIG. 11B, when FAK signal activation was reduced by TINAGL1 knockdown, it was confirmed that the EMT marker Twist increased by CAF co-culture decreased together.

### <Example 6> Confirmation of Expression Level of TINAGL1 in Diffuse Type Gastric Cancer Tissue

The expression level of TINAGL1 protein was observed by Western blot in normal gastric and gastric cancer tissues obtained in pairs from patients with diffuse type gastric cancer.

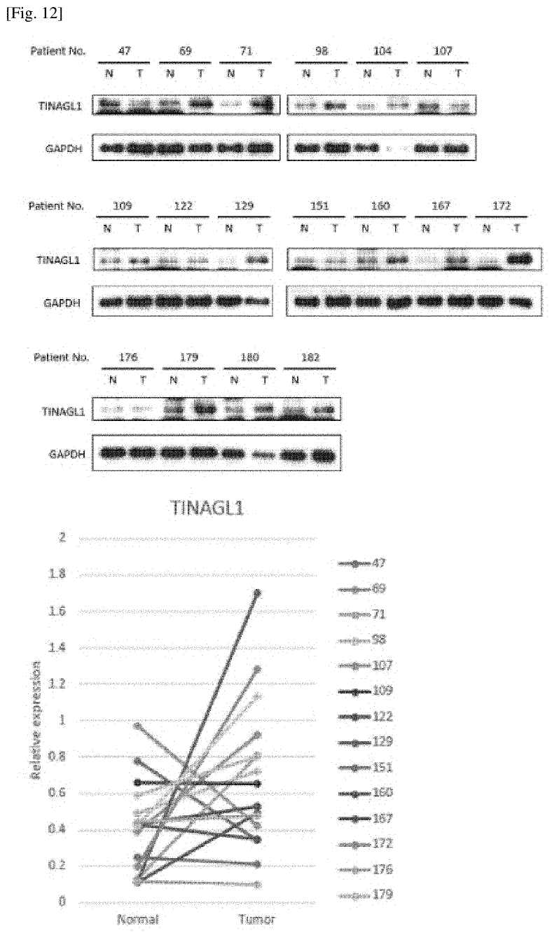

As a result, it was confirmed that TINAGL1 protein expression was higher than that of normal tissues in cancer tissues of most patients with diffuse type gastric cancer, as shown in FIG. 12.

In addition, mRNA expression levels of COL1A1 (red, fibroblast marker) and TINAGL1 (green) were observed in formalin-fixed paraffin-embedded (FFPE) tissues of patients with diffuse type gastric cancer by performing double-staining RNA-in situ hybridization.

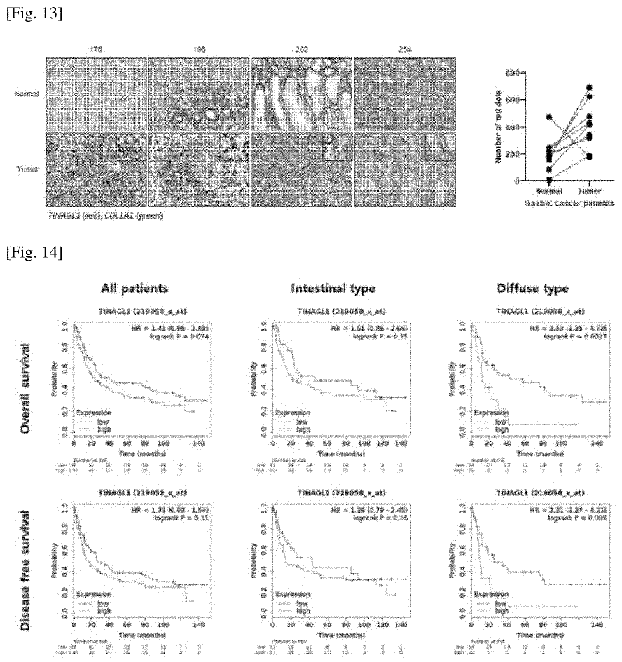

As a result, it was confirmed that TINAGL1 mRNA expression was increased in the tumor tissue, and that it was co-expressed with COL1A1, as shown in FIG. 13.

From the above results, it was confirmed that the expression of TINAGL1 was increased in cancer tissues than in normal gastric tissues.

In addition, association between the TINAGL1 expression level and the prognosis of patients with gastric cancer was confirmed using the published transcriptome data of gastric cancer.

Referring to FIG. 14, high expression of TINAGL1 was not associated with the prognosis of all patients and intestinal type patients, but it was found that high expression of TINAGL1 was significantly associated with poor prognosis in diffuse type gastric cancer with a high stroma ratio.

From the above results, it was confirmed that TINAGL1 gene expression in the stroma is very meaningful in predicting the prognosis of diffuse type gastric cancer.

Although specific parts of the present disclosure have been described in detail above, it is clear for those skilled in the art that these specific descriptions are merely preferred example embodiments and the scope of the present disclosure is not limited thereto. Accordingly, the substantial scope of the present invention will be defined by the appended claims and equivalents thereof.

1-5. (canceled)

6. A biomarker composition for predicting prognosis of diffuse type gastric cancer comprising TINAGL1 protein or a gene encoding the protein as an active ingredient.

7. A pharmaceutical composition for preventing or treating diffuse type gastric cancer comprising a TINAGL1 inhibitor as an active ingredient.

8. The pharmaceutical composition for preventing or treating diffuse type gastric cancer of claim 7, wherein the TINAGL1 inhibitor inhibits secretion and expression of TINAGL1 protein or a gene encoding the protein in cancer-associated fibroblasts (CAF) in diffuse type gastric cancer tissue.

9. The pharmaceutical composition for preventing or treating diffuse type gastric cancer of claim 7, wherein the TINAGL1 inhibitor is any one selected from the group consisting of a peptide, an aptamer, an antibody, a antisense nucleotide, a siRNA, a shRNA, and a miRNA that specifically bind to TINAGL1 protein or a gene encoding the protein.

10. The pharmaceutical composition for preventing or treating diffuse type gastric cancer of claim 7, further comprising an anticancer agent.

11. The pharmaceutical composition for preventing or treating diffuse type gastric cancer of claim 10, wherein the anticancer agent is selected from the group consisting of doxorubicin, paclitaxel, vincristine, daunorubicin, vinblastine, actinomycin-D, docetaxel, etoposide, teniposide, bisantrene, imatinib, cisplatin, and 5-fluorouracil.

12. A drug screening method for treatment of diffuse type gastric cancer comprising:
determining a TINAGL1 expression level by culturing cancer-associated fibroblasts (CAF) isolated from diffuse gastric cancer tissue (step 1);
determining a TINAGL1 expression level by treating the TINAGL1-expressed CAF with a candidate material (step 2); and
comparing the TINAGL1 expression level of step 1 with the TINAGL1 expression level of step 2 (step 3).

13. The drug screening method for treatment of diffuse type gastric cancer of claim 12, wherein the TINAGL1 expression level is measured by any one or more selected from the group consisting of reverse transcription-polymerase chain reaction (RT-PCR), enzyme-linked immunosorbent assay (ELISA), radioimmunoassay, immunohistochemistry, microarray, western blotting, and flow cytometry (FACS).
